(19) United States
(12) Patent Application Publication
(10) Pub. No.: US 2022/0106638 A1
(43) Pub. Date: Apr. 7, 2022

(54) Peptide Nanostructure for Biopolymer Sensing

(71) Applicant: Universal Sequencing Technology Corporation, Canton, MA (US)

(72) Inventors: Peiming Zhang, Gilbert, AZ (US); Ming Lei, Sharon, MA (US)

(21) Appl. No.: 17/429,323

(22) PCT Filed: Feb. 7, 2020
(86) PCT No.: PCT/US20/17359

§ 371 Date: Aug. 6, 2021

Related U.S. Application Data
(60) Provisional application No. 62/803,100, filed on Feb. 8, 2019.

(51) Int. Cl. C12Q 1/6869; G01N 27/327

(52) U.S. Cl. CPC C12Q 1/6869; G01N 27/3278

(57) ABSTRACT

This invention is related to electronic identification and sensing of biomolecules using enzymes incorporated into nanostructures constructed with conductive peptides and/or peptide complexes.

### CROSS-REFERENCE TO RELATED APPLICATION

This application claims priority to U.S. Provisional Application Ser. No. 62/803,100 filed Feb. 8, 2019, the entire disclosure of which is hereby incorporated herein by reference.

### FIELD

Embodiments of the present invention are related to systems, methods, devices, and compositions of matter for electronic sequencing of biopolymers. More specifically, the present invention includes embodiments which teach the construction of a system for detecting biopolymers electronically based on enzymatic replication.

### BACKGROUND OF THE INVENTION

Collins and coworkers devised a method to monitor enzymatic process of synthesizing DNA by a Klenow fragment of DNA polymerase I attached to a single-wall carbon nanotube (SWCNT) field-effect transistor (FET)^{1, 2} In the device, when a nucleotide was incorporated into a DNA strand, a brief excursion of ΔI(t) below the mean baseline currents was recorded. The ΔI signals may be associated with the dynamics of the DNA polymerase in its conformation. Importantly, the characteristics of the signal reflected a specific nucleotide that was incorporated into DNA. It opens a way to read out DNA sequences electronically. As far as the carbon nanotube is concerned, it is a material made from just a single layer of carbon atoms locked in a hexagonal grid. Because of the rigid chemical structure, its sensing may rely on electrostatic gating motions of charged side chains of the enzyme close to the attachment site, which can be shielded by electrolytes in solution. Also, the carbon nanotube in the device had a length of 0.5-1.0 μm,³ which poses a challenge to attaching a single protein molecule of a diameteress than 10 nm to a specific location in such a long wire precisely.

Huang's group reported another type of device with a DNA polymerase grabbed in an antibody bridged nanogap (FIG. 1a).⁴ Electrical fluctuations were recorded as the DNA polymerase elongated a DNA chain by incorporating nucleotides. With the severe controversy, the paper was subsequently retracted (Nat. Nanotechnol. 2015, 10, 563), but it illustrates an idea of monitoring the conformational dynamics of a protein by electrical fluctuation. Besides, the transmission electron microscopy (TEM) image demonstrated that the DNA polymerase laid on the SiO₂ surface out of the nanogap. This configuration would prevent the polymerase from effectively interacting with DNA due to the steric hindrance posed by the surface,

In the other prior art, the invention (WO 2017/024049) provides a nanoscale field-effect transistor (nanoFET) for DNA sequencing, where the DNA polymerase is is immobilized with its nucleotide exit region oriented toward a carbon nanotube gate.

One invention (US 2017/0044605) has claimed an electronic sensor device to sequence DNA and RNA using a polymerase immobilized on a biopolymer that bridges two separate electrodes, In another prior art (US 2018/0305727, WO 2018/208505), a single enzyme is directly wired to both positive and negative electrodes to complete a circuit such that all electrical currents must flow through the molecule. Also, the enzyme is attached to electrodes through more than two contacting points. Nonetheless, it requires a sub-10 nm nanogap, which poses a great challenge to manufacturing.

It has been demonstrated that a protein can become conductive above a bias threshold.⁵ Natural Peptides lose their conductivity quickly because of their relatively flexible conformations.

The ϕ29 DNA polymerase is the only enzyme involved in the replication of the phage ϕ29 genome. Based on amino acid sequence similarities and its sensitivity to specific inhibitors, ϕ29 DNA polymerase belongs to the eukaryotic-type family B of DNA-dependent DNA polymerases (Bernad et al. 1987). As any other DNA polymerase, it accomplishes sequential template-directed addition of dNMP units onto the 3′-OH group of a growing DNA chain, showing discrimination for mismatched dNMP insertion by a factor from 10⁴ to 10⁶ (Esteban et al. 1993). Besides, ϕ29 DNA polymerase catalyzes 3′-5′ exonucleolysis, to release dNMP units from the 3′ end of a DNA strand (Blanco and Salas 1985), degrading preferentially a mismatched primer-terminus, and further enhancing replication fidelity by 10²-fold (Esteban et al, 1994; Garmendia et al. 1992), as it occurs in most DNA replicases.

Three factors that disrupt alfa helix formation: (a) glycine—which is the smallest amino acid; (b) proline—which is the least common amino acid in the alpha-helix which destabilizes it; (c) amino acids with like charge side chains that are close together that are incompatible with the alpha helix.

Molecular self-assembly is ideally suited to create nanostructures with dimensions ranging from 10-100 nm, a size regime suitable for most electronic materials.

It has been reported that a silicon nanowire field-effect transistor (FET) built on the edge of a SiN nanopore can detect DNA translocation by sensing the changes in electrical potential.⁶ Also, a field-effect transistor can sense conformational changes in proximity to semiconductor channels gated conductance in physiological buffers, resulting in the highly sensitive detection of ligand and receptor interactions.⁷ However, these FET devices have not exhibited a capacity to read single DNA bases in a DNA strand.

A simple nanojunction can be formed by connecting a molecular wire to two electrodes separated by a nanoscale gap. It allows electrons to flow when integrated into an electrical circuit. In general, the molecular component is covalently attached to the electrodes, and the electrical conductivity of a junction is affected by the molecular structure and molecule-metal contact.⁸ However, its electronic state can be switched by stereoelectronic effect⁹ and altered by external stimuli. For example, the conductance of a host-guest molecular junction can be tuned by the insertion of guest molecules.¹⁰ Also, a protein transistor can be fabricated by bridging a nanogap using an antigold nanoparticle antibody.¹¹

Electron transfer (ET) can be mediated along with proteins and peptides.^{12, 13} Arguably, ET through peptides may operate through tunneling and hopping in parallel; however, their contributions change with the length of the mediating bridge. For short bridges, tunneling is dominating, whereas for long bridges, hopping becomes more pronounced,¹⁴ which was demonstrated experimentally by Isied and coworkers.¹⁵ The composition of the side chains, hydrogen bonds, and an α-helical secondary structure have been identified as important factors contributing to the hopping and tunneling conductivity in these peptide systems over short distances. Thus, the charge transfer properties of peptides can be modulated by manipulating their secondary structure.

Long-range electron transport in conductive pili represents a natural inspiration for the design of molecular bioelectronics and tunable synthetic platforms for molecular sensing. The proteinaceous pili of Geobacter sulfurreducens can conduct electrons over micrometer distance with metallic-like conductivity.¹⁶ They are unique biological electronic materials. The conductive pili (e-pili) are composed of a single peptide monomer, PilA, which is homologous to the pilin monomer of type IV pili.¹³ In Geobacter sulfurreducens, the major pilin subunit is encoded by the gene pila, which produces the protein PilA with a sequence shown in FIG. 3a. The protein PilA itself is not conductive¹⁷ since it only contain a few of aromatic amino acids residues which are scattered in an a helix (FIGS. 3b & 3c). It has been determined by NMR that the Geobacter sulfurreducens PilA adopts a long, kinked α-helix with a dynamic C-terminal region (FIG. 3, A).¹⁸ Thus, the conductivity of e-pili can be putatively explained by a continuous arrangement of aromatic amino acids in a G. sulfurreducens pilus (FIG. 3, B).¹⁹ It has been demonstrated that aromatic amino acid residues are required for pili conductivity and long-range extracellular electron transport in Geobacter sulfurreducens.²⁰

### BRIEF DESCRIPTION OF THE DRAWINGS

FIG. 1: a prior art of electrical detection system for polymerase fluctuations reported by Chen et al.⁴ (a) schematic description where phi29 DNA polymerase (light blue) is conjugated to a secondary antibody (beige lines) and binds the Fc domain of IgG (blue lines) bearing two gold nanoparticles separately attached to two electrodes to ensure that the antibody assembly is bound into the integrated circuit; (b) Transmission electron microscopy (TEM) image of a phi29-conjugated protein transistor carrying a bound oligonucleotide template and annealed primer.

FIG. 2: A prior art of using biopolymers to connect a DNA polymerase to in electrodes.

FIG. 3: (a) Amino acid sequence of PilA protein of the conductive pilus; (b) PilA protein's α helical model; (c) helical wheel plots of PilA protein.

FIG. 4: (a) a predicted structure of G. sulfurreducens pilin monomers derived from NMR (b) a predicted structure of a Geobacter sulfurreducens pilus derived using monomer pilin based on NMR structure.

FIG. 5: (a) an amino acid sequence of the modified PilA protein; (b) helical wheel plots of the modified PilA protein. (c) the modified PilA protein's α helical model.

FIG. 6: a library of unnatural L-aromatic amino acids for the construction of conductive proteins and peptides.

FIG. 7: a library of unnatural D-aromatic amino acids for the construction of conductive proteins and peptides.

FIG. 8: (a) a three-arm linker for connecting two helical peptides; (b) a coiled coil peptide dimer through one three-arm linker; (c) a peptide timer capped by two three-arm linkers.

FIG. 9: (a) a nanojunction composed of a peptide nanostructure bridging a nanogap; (b) a DNA polymerase immobilized on the nanojunction for DNA sequencing.

FIG. 10: Chemical structures of unnatural amino acids for attachments of peptide nanostructures as well as immobilization of proteins and peptides.

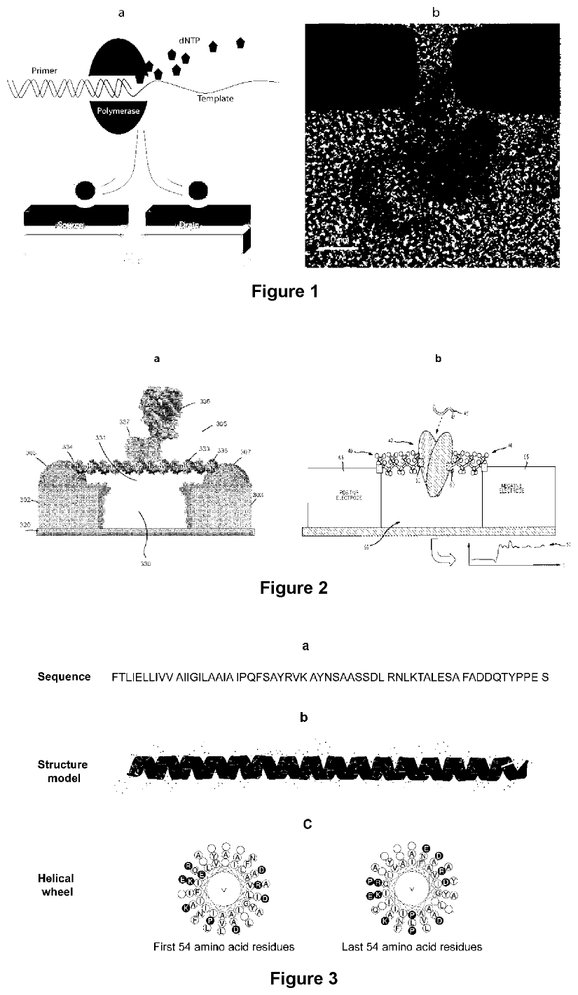

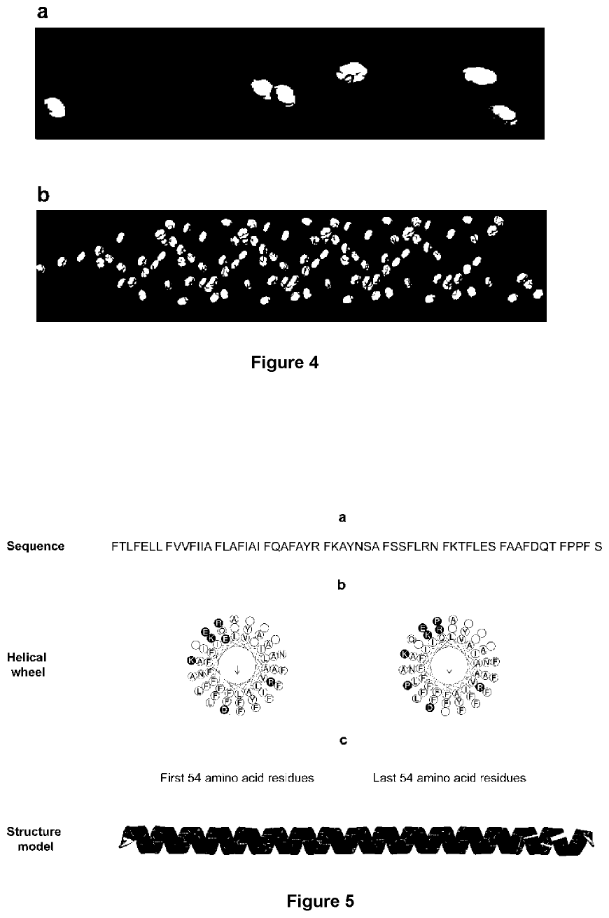

### SUMMARY OF THE INVENTION

One embodiment of this invention provides conductive peptides by modifying the PilA sequence with aromatic amino acids. First, the Pila sequence is rearranged like repeats of a heptad pattern (abcdefg)ₙ where n is the number of in repeats. The aromatic amino acid (F) is substituted for the amino acids at the position a and d of the heptad. As a result, a modified peptide is created with a sequence, as shown in FIG. 5a. The modified peptide can take a helical structure with an aromatic amino acid-rich region as shown in the helical wheel plot (FIG. 5b). The modified peptide has a train of aromatic moieties exposed on its surface is with distances between the aromatic rings smaller than <6 Å (FIG. 5c), which allow electrons to flow through tunneling or hopping, functioning as a molecule wire.

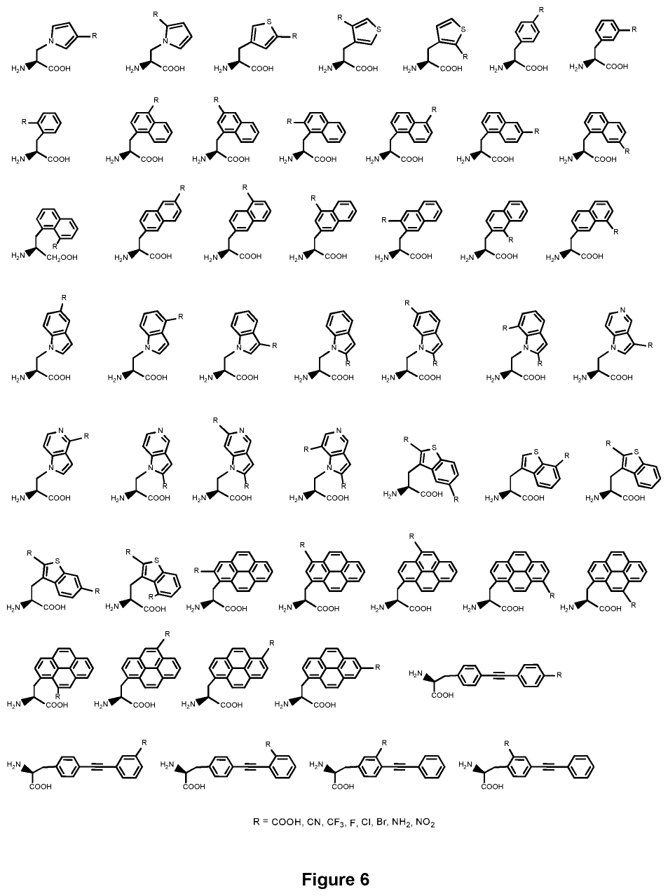

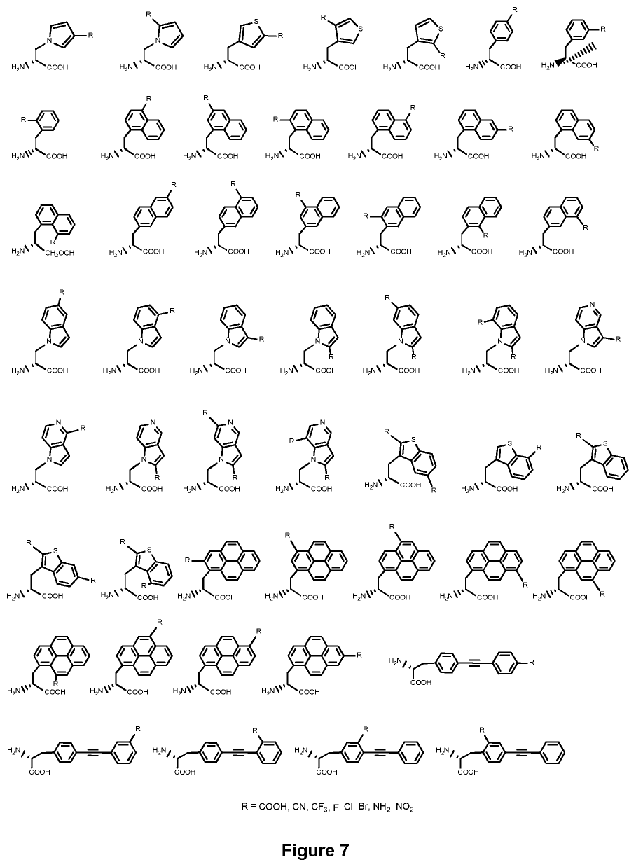

This invention provides also unnatural aromatic amino acids (UAAA) for the construction of conductive proteins and peptides. In one embodiment, it provides a library of UAAAs with an L-configuration (FIG. 6), and in another embodiment, it provides a library of UAAAs with a D-configuration (FIG. 7). UAAAs are incorporated into proteins and peptides by means of bioengineering and/or chemical methods.

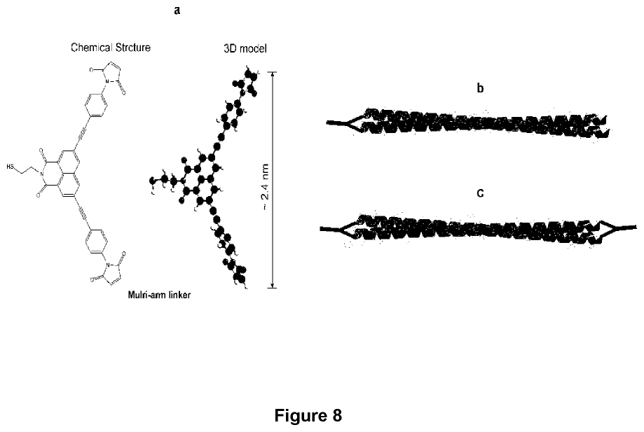

In one embodiment, the invention provides a three-arm linker for the formation of a peptide nanostructure and its attachment to electrodes (FIG. 8a). It also provides a method to make coiled-coil conductive peptides using the three-arm linker (FIG. 8b) and a peptide dimer with its two ends capped by the three-arm linker (FIG. 8c). The peptide nanostructure forms an aromatic tunnel for the electron flow, functioning as a metal-like wire.

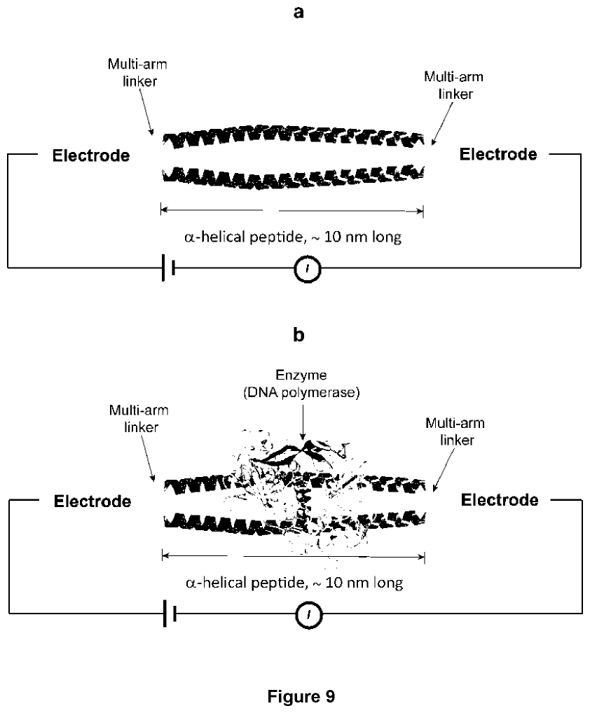

The invention also provides methods to attach the peptide nanostructures to a nanogap composed of electrodes to form a nanojunction for bio- and chemo-sensing (FIG. 9a). In one embodiment, the invention provides a method to immobilize a DNA polymerase to the junction (FIG. 9b) for DNA sequencing.

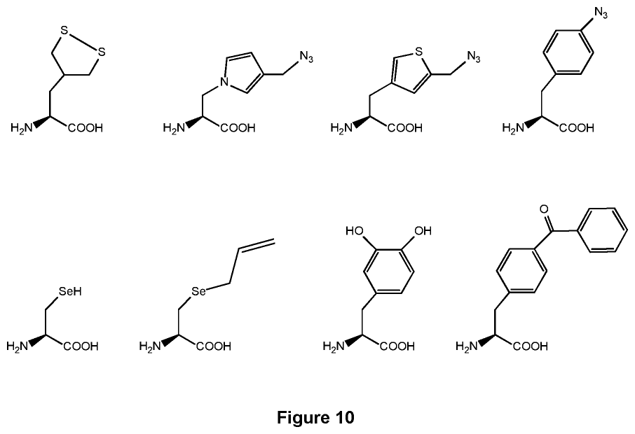

In one embodiment, the invention provides unnatural amino acids for attachment of peptides to electrodes and immobilization of proteins on nanojunctions composed of peptides or peptide nanostructures (FIG. 10) using conjugation chemistry, including but not limited to click chemistry and photochemistry.

Furthermore, this invention discloses the following nanostructures and methods for constructing these nanostructures for electronic sensing, sequencing and/or identification of biomolecules or biopolymers, including but not limited to DNA, RNA, oligos, proteins, peptides, polysaccharides, etc. either natural or modified or synthesized:

1. A system for electronic identification and sequencing of a biopolymer in a is nanogap comprising a first electrode and the second electrode in proximity to said first electrode, which is bridged by a peptide nanostructure bonding to both electrodes through chemical bonds to form nanojunction that does not break over the time course of a measurement process,
2. The said nanojunction in item 1 is functionalized by attaching an enzyme, protein, receptor, nucleic acid probe, antibody and its variants, aptamer, supramolecular host to the nanostructure for the detection of chemical and biochemical reactions as well as molecular interactions.
3. Under a bias applied between the first and second electrodes, the device records current fluctuations resulting from the said nanostructure's distortions caused by the conformation changes of the enzyme attached to the nanostructure while carrying out biochemical reactions. A bias is chosen between the two electrodes so that a steady DC current is observed, and current fluctuations arise when biochemical reactions take place between the said electrodes. In a polymerization reaction, a train of electrical spikes is recorded for the determination of the polymeric sequences.
4. The enzyme in item 1 and item 3 includes but not limited to DNA polymerase, RNA polymerase, DNA helicase, DNA ligase, DNA exonuclease, reverse transcriptase, RNA primase, ribosome, sucrase, lactase, etc., either natural, mutated or synthesized, wherein the DNA polymerase is selected among the group of ϕ29 DNA polymerase, T7 DNA polymerase, Tag polymerase, DNA polymerase Y, DNA Polymerase Pol I, Pol II, Pol III, Pol IV and Pol V, Pol α (alpha), Pol β (beta), Pol σ (sigma), Pol λ (lambda), Pol δ (delta), Pol ε (epsilon), Pol μ (mu), Pol ι (iota), Pol κ (kappa), pol η (eta), terminal deoxynucleotidyl transferase, telomerase, etc., either natural, mutated or synthesized;
5. The said electrodes in item 1 are composed of:

a) metal electrodes that can be functionalized on their surfaces by self-assembling monolayers that can react with anchoring molecules by forming covalent bonds.

b) metal oxide electrodes that can be functionalized with silanes that can react with anchoring molecules to form covalent bonds.

c) carbon electrodes that can be functionalized with organic reagents that can react with anchoring molecules to form covalent bonds.

Wherein metal electrodes include but not limited to Au, Pd, Pt, Cu, Ag, Ti, TiN, or other transition metals.

6. The said nanogap in item 1:

(a) has a length of 3 to 10,000 nm, preferably 5 to 100 nm, and most preferably 5 to 50 nm; a width of 3 to 1000 nm, preferably 10 to 50 nm; and a depth of 2 to 1000 nm, preferably 5 to 50 nm.

(b) is fabricated on substrates including but not limited to glass, silicon and silicon oxide, and polymer films.

7. The said nanostructure in item 1:

(a) is a single peptide chain with helical structure, constructed using a modified bacterial PilA sequence with aromatic amino acid arrangement depicted in FIG. 5 or similar amino acid composition and arrangement;

(b) is a single peptide chain with helical structure, constructed using unnatural aromatic amino acids with either an L-configuration (FIG. 6) or a D-configuration (FIG. 7), or a combination thereof;

(c) is a single peptide/DNA/RNA mixed helical chain constructed using either s natural or modified or synthesized aromatic amino acids and nucleic acids with distance between any two adjacent aromatic rings smaller than 0.6 nm (6 Å), preferably less than 0.35 nm;

(d) is a single peptide coupled with conductive organic conjugates and/or conductive polymers;

(e) is a dual peptide chain consisting of two helical peptide chains either the same composition and arrangement or different composition and arrangement, and with each peptide chain attaching to the electrodes individually or two peptide chain forming a peptide dimer and attaching to the electrodes through a three-arm linker, such as that shown in FIG. 8;

(f) is a peptide chain and a nucleic acid chain forming a dual linear chain structure, helical or non-helical, wherein the peptide chain is made of aromatic amino acids, either natural or synthesized, and the aromatic rings of the amino acids and nucleic acids interacting with each other with distance between any two adjacent rings, either from the peptide chain or from the nucleotide chain, less than 0.6 nm, preferably less than 0.35 nm.

(g) is a multiple peptide chain or a multiple peptide/DNA/RNA mixed chain bundled together forming a two-dimensional nanostructure, or a three-dimensional nanostructure including a bundle of columns, a stack of two-dimensional structures or a folded chain structure such as coiled coils, with a length that can bridge the said two electrodes.

Wherein all the nanostructures mentioned above have a length equivalent to the said nanogap size and able to bridge the two electrodes, and contain functional groups for attachment to electrodes and functional groups for the immobilization of enzymes.
8. The said functional groups for attachment in item 7 include but not limited to:

(a) those thiols on the sugar rings of nucleosides and amino acids.

(b) those thiols and selenols on the nucleobases of nucleosides.

(c) those aliphatic amines on nucleosides.

(d) Those catechols on nucleoside.

(e) azide, alkyne and alkene on unnatural amino acids.

(f) Photoactive groups such as benzophenone

9. The said anchoring molecules in item 5 are

(a) those that can interact with the metal surface through multivalent bonds.

(b) a tripod structure that can interact with the metal surface through trivalent bonds.

(c) Those that are composed of a tetraphenylmethane core of which three phenyl rings are functionalized with —CH₂SH and —CH₂SeH and the last phenyl ring is functionalized with azide, carboxylic acid, boronic acid, and organic groups that can react with those functional groups incorporated into peptide, DNA and RNA nanostructures.

10. The said anchoring molecules in item 5 are

(a) N-heterocyclic carbenes (NHC);

(b) N-heterocyclic carbenes (NHC) that are selectively deposited to cathode electrodes by electrochemical methods with their metal complexes in solutions.

(c) N-heterocyclic carbenes (NHC) that are deposited to both metal electrodes in organic and aqueous solutions.

(d) N-heterocyclic carbenes (NHC) containing functional groups including amines, carboxylic acids, thiol, boronic acids, or other organic groups for attachment.

11. The said NHC metal complexes in item 10 include but not limited to those composed of Au, Pd, Pt, Cu, Ag, Ti, TiN, or other transition metals or a combination thereof.
12. The said nanogap in item 6 is functionalized with chemical reagents on its bottom.
13. The said chemical reagent in item 12 is:

(a) Silanes that can react with oxide surfaces;

(b) Silatranes that can react with oxide surfaces;

(c) A multi-arm linker that contains silatranes and functional groups;

(d) A four-arm linker that is composed of an adamantane core;

(e) A four-arm linker that contains two silatranes and two biotin moieties.

(f) A four-arm linker that is composed of adamantane core and silatranes and biotin

14. The said chemical reagents in item 12 are used to immobilize proteins in the nanogap, which include antibodies, receptors, streptavidin, avidin.
15. The said streptavidin in item 14 is used to immobilized the said nanostructures.
16. The said nanostructure in item1 is functionalized with biotins.
17. The system of item 1 can contain a single nanogap or a plurality of nanogaps, each with a pair of electrodes, an enzyme, a peptide nanostructure and all other features associated with a single nanogap. Furthermore, the system can consist of an array of nanogaps between 100 to 100 million, preferably between 10,000 to 1 million.
18. The said nanostructure in item 1 is in general conductive by itself. However, in some special cases, it can be made non-conductive by itself but conductive when combines with the enzyme or at least during a portion of the chemical reaction process of the enzyme.
19. Features of nanostructure, nanogap, enzyme and electrode, their composition and construction as well as other associated features and methods mentioned in our provisional application Ser. No. 62/794,096 that are relevant to this invention and can apply to the said nanostructure in this invention are included here in its entirety

### General Remarks

All publications, patents and other documents mentioned herein are incorporated by reference in their entirety.

Unless defined otherwise, all technical and scientific terms used herein have the same meaning as those commonly understood by one of ordinary skill in the art to which this invention belongs. While the present invention has been illustrated by a description of various embodiments and while these embodiments have been described in considerable detail, it is not the intention of the applicants to restrict or in any way limit the scope of the applications. Additional advantages and modifications will readily appear to those skilled in the art. The invention in its broader aspects is therefore not limited to the specific details, representative device, apparatus and method, and illustrative example shown and described. Accordingly, departures may be made from such details without departing from the spirit of applicant's general inventive concept. Finally, the use of singular articles such as “a,” “the,” “said,” etc. should be read to recite one or more of the indicated elements unless a claim recites an explicit limitation to the contrary.

### REFERENCES

1. Olsen, T. J.; Choi, Y.; Sims, P. C.; Gul, O. T.; Corso, B. L.; Dong, C.; Brown, W. A.; Collins, P. G.; Weiss, G. A., Electronic measurements of single-molecule processing by DNA polymerase I (Klenow fragment). J Am Chem Soc 2013, 135 (21), 7855-60.
2. Pugliese, K. M.; Gul, O. T.; Choi, Y.; Olsen, T. J.; Sims, P. C.; Collins, P. G.; Weiss, G. A., Processive Incorporation of Deoxynucleoside Triphosphate Analogs by Single-Molecule DNA Polymerase (Klenow Fragment) Nanocircuits. J Am Chem Soc 2015, 137 (30), 9587-94.
3. Choi, Y.; Olsen, T. J.; Sims, P. C.; Moody, I. S.; Corso, B. L; Dang, M. N.; Weiss, G. A.; Collins, P. G., Dissecting Single-Molecule Signal Transduction in Carbon Nanotube Circuits with Protein Engineering. Nano Lett. 2013, 13, 625-631.
4. Chen, Y. S.; Lee, C. H.; Hung, M. Y.; Pan, H. A.; Chiou, J. C.; Huang, G. S., DNA sequencing using electrical conductance measurements of a DNA polymerase. Nat Nanotechnol 2013, 8 (6), 452-458.
5. Zhang, B.; Song, W.; Pang, P.; Zhao, Y.; Zhang, P.; Csabai, I.; Vattay, G.; Lindsay, S., Observation of Giant Conductance Fluctuations in a Protein. Nano Futures 2017, 1 (3), 035002.
6. Xie, P.; Xiong, Q.; Fang, Y.; Qing, Q.; Lieber, C. M., Local electrical potential detection of DNA by nanowire-nanopore sensors. Nature Nanotechnology 2012, 7, 119-125.
7. Nakatsuka, N.; Yang, K.-A.; Abendroth, J. M.; Cheung, K. M.; Xu, X.; Yang, H.; Zhao, C.; Zhu, B.; Rim, Y. S.; Yang, Y.; Weiss, P. S.; Stojanović, M. N.; Andrews, A. M., Aptamer-field-effect transistors overcome Debye length limitations for small-molecule sensing. Science 2018, 362, 319-324.
8. Wang, G.; Kim, T.-W.; Jang, Y. H.; Lee, T., Effects of Metal-Molecule Contact and Molecular Structure on Molecular Electronic Conduction in Nonresonant Tunneling Regime: Alkyl versus Conjugated Molecules. J. Phys. Chem, C 2008, 112, 13010-13016,
9. Xin, N.; Wang, J.; Jia, C.; Liu, Z.; Zhang, X.; Yu, C.; Li, M.; Wang, S.; Gong, Y.; Sun, H.; Zhang, G.; Liu, Z.; Zhang, G.; Liao, J.; Zhang, D.; Guo, X., Stereoelectronic Effect-Induced Conductance Switching in Aromatic Chain Single-Molecule Junctions. Nano Lett. 2017, 17, 856-861.
10. Fujii, S.; Tada, T.; Komoto, Y.; Osuga, t; Murase, T.; Fujita, M.; Kiguchi, M., Rectifying Electron-Transport Properties through Stacks of Aromatic Molecules Inserted into a Self-Assembled Cage. J Am Chem Soc 2015, 137 (18), 5939-47.
11. Chen, Y.-S.; Hong, M.-Y.; Huang, G. S., A protein transistor made of an antibody molecule and two gold nanoparticles. Nature Nanotechnology 2012, 7, 197-203.
12. Amdursky, N., Electron Transfer across Helical Peptides. ChemPlusChem 2015, 80(7), 1075-1095.
13. Reguera, G.; McCarthy, K. D.; Mehta, T.; Nicoll, J. S.; Tuominen, M. T.; Lovley, D. R., Extracellular electron transfer via microbial nanowires. Nature 2005, 435 (7045), 1098-101.
14. Petrov, E. G.; Shevchenko, Y. V.; Teslenko, V. I.; May, V., Nonadiabatic donor-acceptor electron transfer mediated by a molecular bridge: A unified theoretical description of the superexchange and hopping mechanism. The Journal of chemical physics 2001, 115 (15), 7107-7122.
15. Malak, R. A.; Gao, Z.; Wishart, J. F.; !sled, S. S., Long-Range Electron Transfer Across Peptide Bridges: The Transition from Electron Superexchange to Hopping. J. AM. CHEM. SOC. 2004, 126, 13888-13889
16. Lovley, D, R., e-Biologics: Fabrication of Sustainable Electronics with “Green” Biological Materials. mBio 2017, 8 (3).
17. Lebedev, N.; Mahmud, S.; Griva, I.; Blom, A.; Tender, L. M., On the Electron Transfer through Geobacter sulfurreducens PilA Protein. JOURNAL OF POLYMER SCIENCE, PART B: POLYMER PHYSICS 2015, 53, 1706-1717.
18. Reardon, P. N.; Mueller, K. T., Structure of the type IVa major pilin from the electrically conductive bacterial nanowires of Geobacter sulfurreducens. J Biol Chem 2013, 288 (41), 29260-6.
19. Malvankar, N. S.; Vargas, M.; Nevin, K.; Tremblay, P. L.; Evans-Lutterodt, K.; Nykypanchuk, D.; Martz, E.; Tuominen, M. T.; Lovley, D. R., Structural basis for metallic-like conductivity in microbial nanowires. mBio 2015, 6 (2), e00084.
20. Vargas, M.; Malvankar, N. S.; Tremblay, P. L,; Leang, C.; Smith, J. A.; Patel, P.; Synoeyenbos-West, O.; Nevin, K. P.; Lovley, D. R., Aromatic Amino Acids Required for Pili Conductivity and Long-Range Extracellular Electron Transport in Geobacter sulfurreducens. mBio 2013, 4 (2).

1. A system for identification, characterization, or sequencing of a biopolymer comprising,
(a) a non-conductive substrate, either comprising non-conductive materialor coated with non-conductive material;
(b) a nanogap formed by a first electrode and a second electrode placed next to each other on the non-conductive substrate;
(c) a peptide nanostructure configured to bridge the said nanogap by la attaching one end to the first electrode and another end to the second electrode through chemical bonds, wherein the peptide nanostructure is conductive;
(d) an enzyme attached to the peptide nanostructure configured to perform a biochemical reaction and/or snesing;
(e) a bias voltage that is applied between the first electrode and the second electrode;
(f) a device configured to record an electrical signal fluctuation in the peptide nanostructure resulting from a distortion within the peptide nanostructure caused by a conformation change of the peptide nanostructure initiated by the enzyme; and
(g) a software configured for data analysis that identifies or characterizes the biopolymer or a subunit of the biopolymer.

2. The system of claim 1, wherein the non-conductive material comprises the group consisting of silicon, silicon oxide, silicon nitride, glass, hafnium dioxide, metal oxide, non-conductive polymer film, any non-conductive organic material, any non-conductive inorganic material, and the combination thereof;

3. The system of claim 1, wherein the biopolymer is selected from the group consisting of DNA, RNA, oligonucleotides, protein, peptides, polysaccharides , either natural, modified or synthesized of any of the aforementioned biopolymers, and a combination thereof.

4. The system of claim 1, wherein the enzyme is selected from the group consisting of DNA polymerase, RNA polymerase, DNA helicase, DNA ligase, DNA exonuclease, reverse transcriptase, RNA primase, ribosome, sucrase, lactase, either native, mutated, expressed, or synthesized of any of the aforementioned enzymes, and a combination thereof.

5. The system of claim 4, wherein the DNA polymerase is selected from the group consisting of T7 DNA polymerase, Tag polymerase, DNA polymerase Y, DNA Polymerase Pol I, Pol II, Pol III, Pol IV and Pol V, Pol α (alpha), Pol β (beta), Pol σ (sigma), Pol λ (lambda), Pol δ (delta), Pol ε (epsilon), Pol μ (mu), Pol I (iota), Pol κ (kappa), pol η (eta), terminal deoxynucleotidyl transferase, telomerase, either native, mutated, expressed, or synthesized, and a combination thereof

6. The system of claim 4, wherein the DNA polymerase is Phi29 (ϕ29) DNA polymerase, either native, mutated, expressed, or synthesized.

7. The system of claim 1, wherein when the electrodes have substantially a is rectangular conformation,
the said nanogap has a length (distance separating the two electrodes) of about 3 nm to about 10,000 nm, a width (width of the electrodes) of about 3 nm to about 1000 nm, and a depth (thickness of the electrodes) of about 2 nm to about 1000 nm.

8. The system of claim 1, wherein when the electrodes have substantially a rectangular conformation,
the said nanogap has a length (distance separating the two electrodes) of about 5 nm to about 100 nm, a width (width of the electrodes) of about 10 nm to about 50 nm, and a depth (thickness of the electrodes) of about 5 nm to about 50 nm.

9. The system of claim 1, wherein the said electrodes are comprised of:
d) a metal electrode that can be functionalized on its surface by self-assembling monolayers that is configured to react with an anchoring molecule by forming a covalent bond;
e) a metal oxide electrode that can be functionalized with silanes that is configured to react with an anchoring molecule to form a covalent bond; and/or
f) a carbon electrode that can be functionalized with an organic reagent configured to react with an anchoring molecule to form a covalent bond.

10. The system of claim 9, wherein the anchoring molecule comprises the following:
a. a molecule with a thiol group;
b. a molecule with a selenol group;
c. a molecule with an aliphatic amine group;
d. a molecule with a catechol group;
e. a molecule with an azide, alkyne or alkene group; and/or
f. a photoactive group, such as benzophenone.

11. The system of claim 9, wherein the anchoring molecule comprises at least one of the following or a combination thereof:
a. a N-heterocyclic carbene (NHC);
b. a N-heterocyclic carbene (N HC) that is selectively deposited to a cathode electrode by electrochemical method with a metal complexe in solution, wherein the metal complex comprises Au, Pd, Pt, Cu, Ag, Ti, or TiN, or another transition metal or a combination thereof;
c, a N-heterocyclic carbene (N HC) that is deposited to both metal electrodes in an organic and/or aqueous solution; and
d. a N-heterocyclic carbene (N HC) containing a functional group comprising an amine, a carboxylic acid, a thiol, a boronic acid, or another organic group for attachment, or the combination thereof.

12. The system of claim 1, wherein the electrode is a metal electrode, comprising Au, Pd, Pt, Cu, Ag, Ti, TiN, or another transition metal, or a combination thereof.

13. The system of claim 1, wherein the peptide nanostructure comprises at least one of the following or a combination thereof:
a. a single peptide chain with helical structure, constructed using a modified bacterial PilA sequence with aromatic amino acid arrangement or a substantially similar amino acid composition and arrangement;
b. a single peptide chain with helical structure, constructed using unnatural aromatic amino acids with either an L-configuration (FIG. 6) or a D-configuration, or a combination thereof;
c. a single peptide/DNA/RNA mixed helical chain, constructed using either a natural or a modified or a synthesized aromatic amino acid la and/or nucleic acid with a distance between any two adjacent aromatic rings less than 0.6 nm;
d. a single peptide coupled with a conductive organic conjugate and/or a conductive polymer;
e. a dual peptide chain comprising two helical peptide chains either the is same composition and arrangement or different composition and arrangement, and with each peptide chain attached to the electrodes individually or two peptide chain forming a peptide dimer and attached to the electrodes through a three-arm linker;
f. a peptide chain and a nucleic acid chain forming a dual linear chain structure, either helical or non-helical, wherein the peptide chain comprises an aromatic amino acid, either natural or synthesized, and the aromatic ring of the amino acid and the aromatic ring of the nucleic acid interact with each other at a distance, wherein the distance between any two adjacent aromatic rings, either from the peptide chain or from the nucleotide chain, is less than about 0.6 nm; and
g. a multiple peptide chain or a multiple peptide/DNA/RNA mixed chain bundled together forming a substantially two-dimensional nanostructure, or a substantially three-dimensional nanostructure comprising a bundle of columns, a stack of two-dimensional structures or a folded chain structure such as coiled coils, with a length configured to bridge the two electrodes.

14. The system of claim 13, wherein for the peptide nanostructure comprised of a mixture of amino acids and nucleotides, the distance between any two adjacent aromatic rings, either from an amino acid or a nucleotide, is less than about 0.35 nm.

15. The system of claim 1, wherein the peptide nanostructure has an approximate length equivalent to the nanogap size and is configured to bridge the two electrodes, and comprises a functional group for attachment to the electrode and la a functional group to immobilize the enzyme.

16. The system of claim 15, wherein the said functional group for the attachment to the electrode comprises at least one of the following:
a. a thiol on the sugar ring of a nucleoside and/or an amino acid,
b. a thiol and a selenol on a nucleobase of a nucleoside,
c. an aliphatic amine on a nucleoside,
d. a catechol on a nucleoside,
e. an azide, an alkyne and/or an alkene on an unnatural amino acid, and/or
f. a photoactive group, such as a benzophenone.

17. The system of claim 15, wherein the said functional group for the attachment to the electrode comprises at least one of the following:
a. a tripod (four-arm linker) structure configured to interact with the metal surface through trivalent bonds; and/or
b. a molecule comprised of a tetraphenylmethane core of which three phenyl rings are functionalized with —CH₂SH and —CH₂SeH and the fourth phenyl ring is functionalized with an azide, a carboxylic acid, a boronic acid, and/or an organic group that is configured to react with a functional group incorporated into the peptide nanostructure.

18. The system of claim 1, further comprising:
a protein configured to be immobilized at the non-conductive substrate floor of the nanogap to support and stabilize the peptide nanostructure.

19. The system of claim 18, wherein
the non-conductive floor of the nanogap is functionalized with a chemical reagent configured to immobilize proteins, wherein the chemical reagent comprises at least one of the following or a combination thereof:
(g) a silane configured to react with an oxide surface;
(h) a silatrane configured to react with an oxide surface;
(i) a multi-arm linker that comprises a silatrane and a functional group;
(j) a four-arm linker that comprises an adamantane core;
(k) a four-arm linker that comprises two silatranes and two biotin moieties; and/or
(l) a four-arm linker that comprises an adamantane core and a silatrane and a biotin.

20. The system of claim 18, wherein the protein is selected from the group consisting of an antibody, a receptor, an aptamer, a streptavidin, or an avidin or a combination thereof.

21. The system of claim 20, wherein the streptavidin is configured to immobilized the peptide nanostructure, wherein the peptide nanostructure comprises a biotin.

22. The system of claim 1, wherein the peptide nanostructure is non-conductive but is configured to be conductive when combined with the enzyme during a portion or a whole portion of the enzyme's activity.

23. The system of claim 1, wherein the enzyme is a recombinant DNA polymerase or a recombinant reverse transcriptase that comprises an orthogonal functional group configured to attach the enzyme to the peptide nanostructure.

24. The system of claim 23, wherein the recombinant DNA polymerase or the recombinant reverse transcriptase comprises one of the following or a combination thereof:
(a) an organic group at an N- and/or C-terminal configured for a click reaction on the peptide nanostructure;
(b) an unnatural, modified or synthetic amino acids configured for a click reaction on the peptide nanostructure;
(c) an azide group at an N- and/or C-terminal configured for a click reaction on the peptide nanostructure; and
(d) a 2-amino-6-azidohexanoic acid (6-azido-L-lysine) configured for a click reaction on the peptide nanostructure.

25. The system of claim 1, wherein the biochemical reaction comprises:
(a) a reaction catalyzed by a DNA polymerase using a DNA as a template and a DNA nucleotide as a substrate; and/or
(b) a reaction catalyzed by a reverse transcriptase using a RNA as a template and a DNA nucleotide as a substrate.

26. The system of claim 25, wherein the DNA nucleotide comprises one of the following or a combination thereof:
(a) a DNA nucleoside polyphosphate;
(b) a DNA nucleoside polyphosphate tagged with an organic molecule;
(c) a DNA nucleoside polyphosphate tagged with an intercalator;
(d) a DNA nucleoside polyphosphate tagged with a minor groove binder; and
(e) a DNA nucleoside polyphosphate tagged with a drug molecule.

27. The system of claim 1, wherein the nanogap comprises a plurality of nanogaps, each comprising a pair of electrodes, an enzyme, a peptide nanostructure and any feature associated with a single nanogap.

28. The system of claim 27, wherein the plurality of nanogaps form an array of nanogaps between about 100 to about 100 million nanogaps.

29. The system of claim 27, wherein the plurality of nanogaps form an array of nanogaps between about 1000 to about 1 million nanogaps.

30. A method for identification, characterization, or sequencing of a biopolymer comprising,
(a) providing a non-conductive substrate, either comprising non-conductive material or coated with non-conductive material;
(b) building a nanogap by placing a first electrode and a second electrode next to each other on the non-conductive substrate;
(c) providing a peptide nanostructure that bridges the nanogap by attaching one end to the first electrode and another end to the second electrode through chemical bonds, wherein the peptide nanostructure is conductive;
(d) attaching an enzyme to the peptide nanostructure configured to perform a biochemical reaction and/or sensing, or alternatively, attaching the enzyme to the peptide nanostructure before attaching the peptide la nanostructure to the electrodes that form the nanogap;
(e) applying a bias voltage between the first electrode and the second electrode;
(f) providing a device configured to record an electrical signal fluctuation in the peptide nanostructure resulting from a distortion within the peptide nanostructure caused by a conformation change initiated by the enzyme; and
(g) providing a software configured for data analysis that identifies and/or characterizes the biopolymer or a subunit of the biopolymer.

31. The method of claim 30, wherein the non-conductive material comprises the group consisting of silicon, silicon oxide, silicon nitride, glass, hafnium dioxide, metal oxide, non-conductive polymer film, any non-conductive organic material, any non-conductive inorganic material, and the combination or composite thereof;

32. The method of claim 30, wherein the biopolymer is selected from the group consisting of DNA, RNA, oligonucleotides, protein, peptides, polysaccharides , either natural, modified or synthesized, and a combination thereof.

33. The method of claim 30, wherein the enzyme is selected from the group consisting of DNA polymerase, RNA polymerase, DNA helicase, DNA ligase, DNA exonuclease, reverse transcriptase, RNA primase, ribosome, sucrase, lactase, either native, mutated, expressed, or synthesized of any of the aforementioned enzymes, and a combination thereof.

34. The method of claim 30, wherein the DNA polymerase is selected from the group consisting of T7 DNA polymerase, Tag polymerase, DNA polymerase Y, DNA Polymerase Pol I, Pol II, Pol III, Pol IV and Pol V, Pol α (alpha), Pol β (beta), Pol σ (sigma), Pol λ (lambda), Pol δ (delta), Pol ε (epsilon), Pol μ (mu), Pol I (iota), Pol κ (kappa), pol η (eta), terminal deoxynucleotidyl transferase, telomerase, either native, mutated, expressed, or synthesized, and a combination thereof .

35. The method of claim 30, wherein the DNA polymerase is Phi29 (ϕ29) DNA polymerase, either native, mutated, expressed, or synthesized.

36. The method of claim 30, wherein
the nanogap has a length (distance separating the two electrodes) of about 3 nm to about 10,000 nm, a width (width of the electrodes) of about 3 nm to about 1000 nm, and a depth (thickness of the electrodes) of about 2 nm to about 1000 nm.

37. The method of claim 30, wherein
the nanogap has a length (distance separating the two electrodes) of about 5 nm to about 100 nm, a width (width of the electrodes) of about 10 nm to about 50 nm, and a depth (thickness of the electrodes) of about 5 to about 50 nm.

38. The method of claim 30, wherein the electrode is comprised of:
(a) a metal electrode that can be functionalized on its surface by self-assembling monolayers that are configured to react with an anchoring molecule by forming a covalent bond;
(b) a metal oxide electrode that can be functionalized with silanes configured to react with an anchoring molecule to form a covalent bond; and/or
(c) a carbon electrode that can be functionalized with organic reagents configured to react with an anchoring molecule to form a covalent bond.

39. The method of claim 38, wherein the anchoring molecule comprises at least one of the following or a combination thereof:
a. a molecule with a thiol group,
b. a molecule with a selenol group,
c. a molecule with an aliphatic amine group,
d. a molecule with a catechol group,
e. a molecule with either an azide, an alkyne and/or an alkene group, and/or
f. a photoactive group, such as a benzophenone. la 40.The method of claim 38, wherein the said anchoring molecule comprises at least one of the following or a combination thereof:
a. a N-heterocyclic carbene (NHC);
b. a N-heterocyclic carbene (NHC) that is selectively deposited to a cathode electrode by electrochemical method with a metal complexe in is solution, wherein the metal complex comprises Au, Pd, Pt, Cu, Ag, Ti, or TiN, or another transition metal or a combination thereof;
c. a N-heterocyclic carbene (NHC) that is deposited to both metal electrodes in an organic and/or aqueous solution; and
d. a N-heterocyclic carbene (NHC) containing a functional group comprising an amine, a carboxylic acid, a thiol, a boronic acid, or another organic group for attachment, or the combination thereof.

41. The method of claim 30, wherein the electrode is a metal electrode, comprising Au, Pd, Pt, Cu, Ag, Ti, TiN, or another transition metal.

42. The method of claim 30, wherein the peptide nanostructure comprises at least one of the following or a combination thereof:
a. a single peptide chain with helical structure, constructed using a modified bacterial PilA sequence with aromatic amino acid arrangement or a substantially similar amino acid composition and arrangement;
b. a single peptide chain with helical structure, constructed using unnatural aromatic amino acids with either an L-configuration (FIG. 6) or a D-configuration, or a combination thereof;
c. a single peptide/DNA/RNA mixed helical chain, constructed using either a natural or a modified or a synthesized aromatic amino acid and/or nucleic acid with a distance between any two adjacent aromatic rings less than 0.6 nm;
d. a single peptide coupled with a conductive organic conjugate and/or a conductive polymer;
e. a dual peptide chain comprising two helical peptide chains either the same composition and arrangement or different composition and arrangement, and with each peptide chain attached to the electrodes individually or two peptide chain forming a peptide dimer and attached to the electrodes through a three-arm linker;
f. a peptide chain and a nucleic acid chain forming a dual linear chain structure, either helical or non-helical, wherein the peptide chain comprises an aromatic amino acid, either natural or synthesized, and the aromatic ring of the amino acid and the aromatic ring of the nucleic acid interact with each other at a distance, wherein the distance between any two adjacent aromatic rings, either from the peptide chain or from the nucleotide chain, is less than about 0.6 nm; and
g. a multiple peptide chain or a multiple peptide/DNA/RNA mixed chain bundled together forming a substantially two-dimensional nanostructure, or a substantially three-dimensional nanostructure comprising a bundle of columns, a stack of two-dimensional structures or a folded chain structure such as coiled coils, with a length configured to bridge the two electrodes.

43. The method of claim 42, wherein for the peptide nanostructure comprised of a mixture of amino acids and nucleotides, the distance between any two adjacent aromatic rings, either from an amino acid or a nucleotide, is less than about 0.35 nm.

44. The method of claim 30, wherein the peptide nanostructure has an approximate length equivalent to the nanogap size and is configured to bridge the two electrodes, and comprises a functional group for attachment to the electrode and a functional group to immobilize the enzyme.

45. The method of claim 44, wherein the said functional group for the attachment to the electrode comprises at least one of the following or a combination thereof:
a. a thiol on the sugar ring of a nucleoside and/or an amino acid,
b. a thiol and a selenol on a nucleobase of a nucleoside,
c. an aliphatic amine on a nucleoside,
d. a catechol on a nucleoside,
e. an azide, an alkyne and/or an alkene on an unnatural amino acid, and/or
f. a photoactive group, such as a benzophenone.

46. The method of claim 44, wherein the said functional group for the attachment to the electrode comprises at least one of the following:
a. a tripod (four-arm linker) structure configured to interact with the metal surface through trivalent bonds; and/or
b. a molecule comprised of a tetraphenylmethane core of which three phenyl rings are functionalized with —CH₂SH and —CH₂SeH and the fourth phenyl ring is functionalized with an azide, a carboxylic acid, a boronic acid, and/or an organic group that is configured to react with a functional group incorporated into the peptide nanostructure.

47. The method of claim 30, further comprising:
providing a protein configured to be immobilized at the non-conductive substrate floor of the nanogap to support and stabilize the peptide nano structure.

48. The method of claim 47, wherein
the non-conductive floor of the nanogap is functionalized with a chemical reagent configured to immobilize proteins, wherein the chemical reagent comprises at least one of the following or a combination thereof:
(m) a silane configured to react with an oxide surface;
(n) a silatrane configured to react with an oxide surface;
(o) a multi-arm linker that comprises a silatrane and a functional group;
(p) a four-arm linker that comprises an adamantane core;
(q) a four-arm linker that comprises two silatranes and two biotin moieties; and/or
(r) a four-arm linker that comprises an adamantane core and a silatrane and a biotin.

49. The method of claim 47, wherein the protein is selected from the group consisting of an antibody, a receptor, an aptamer, a streptavidin, or an avidin or a combination thereof.

50. The method of claim 49, wherein the streptavidin is configured to immobilized the peptide nanostructure, wherein the peptide nanostructure comprises a biotin.

51. The method of claim 30, wherein the peptide nanostructure is non-conductive but is configured to be conductive when combined with the enzyme during a portion or a whole portion of the enzyme's activity.

52. The method of claim 30, wherein the enzyme is a recombinant DNA polymerase or a recombinant reverse transcriptase that comprises an orthogonal functional group configured to attach the enzyme to the peptide nanostructure.

53. The method of claim 52, wherein the recombinant DNA polymerase or the recombinant reverse transcriptase comprises one of the following or a combination thereof:
(e) an organic group at an N- and/or C-terminal configured for a click reaction on the peptide nanostructure;
(f) an unnatural, modified or synthetic amino acids configured for a click reaction on the peptide nanostructure;
(g) an azide group at an N- and/or C-terminal configured for a click reaction on the peptide nanostructure; and
(h) a 2-amino-6-azidohexanoic acid (6-azido-L-lysine) configured for a click reaction on the peptide nanostructure.

54. The method of claim 30, wherein the biochemical reaction comprises:
(c) a reaction catalyzed by a DNA polymerase using a DNA as a template and a DNA nucleotide as a substrate; and/or
(d) a reaction catalyzed by a reverse transcriptase using a RNA as a template and a DNA nucleotide as a substrate.

55. The method of claim 54, wherein the DNA nucleotide comprises one of the following or a combination thereof:
(a) a DNA nucleoside polyphosphate;
(b) a DNA nucleoside polyphosphate tagged with an organic molecule;
(c) a DNA nucleoside polyphosphate tagged with an intercalator;
(d) a DNA nucleoside polyphosphate tagged with a minor groove binder; and
(e) a DNA nucleoside polyphosphate tagged with a drug molecule.

56. The method of claim 30, wherein the nanogap comprises a plurality of nanogaps, each comprising a pair of electrodes, an enzyme, a peptide nanostructure and any feature associated with a single nanogap.

57. The method of claim 56, wherein the plurality of nanogaps form an array of nanogaps between about 100 to about 100 million nanogaps.

58. The method of claim 56, wherein the plurality of nanogaps form an array of nanogaps between about 1000 to about 1 million nanogaps
